(19) United States
(12) Patent Application Publication
(10) Pub. No.: US 2002/0012909 A1
(43) Pub. Date: Jan. 31, 2002

(54) Small functional units of antibody heavy chain variable regions

(76) Inventor: Daniel Plaksin, Rehovot (IL)

Correspondence Address: WINSTON & STRAWN, 200 PARK AVENUE, NEW YORK, NY, 10166-4193, US

(21) Appl. No.: 09/858,349

(22) Filed: May 15, 2001

Related U.S. Application Data
(63) Continuation of application No. PCT/IL99/00581, filed on Nov. 2, 1999.

(30) Foreign Application Priority Data
Nov. 11, 1998 (IL) 127127

(51) Int. Cl. C12Q 1/70; C12N 7/00; A61K 39/395

(52) U.S. Cl. 435/5; 435/235.1; 424/145.1; 424/85.1

(57) ABSTRACT

The present invention relates to small antibody-derived recognition units for experimental, medical, and drug design purposes. A single-domain VH phage-displayed library that is based on a natural framework scaffold of a mouse monoclonal antibody with a unique VH/VL interface and a randomized CDR3 was generated. This library was displayed without any mutations or modifications in the original interface framework residues. The library was used to select phage clones that bind specifically to protein antigens with affinity in the nanomolar range. The VH domains were subsequently produced as soluble proteins at very high yields by expressing them in E. Coli as insoluble inclusion bodies and in-vitro refolding. These small functional modules of antibodies were termed “Microbodies” and were fully characterized by means of biochemical and biophysical properties as well as binding properties to several antigens of interest.

### CROSS-REFERENCE TO RELATED APPLICATIONS

This application is a continuation of the U.S. national stage designation of International Application PCT/IL99/00851 filed Nov. 2, 1999, the content of which is expressly incorporated herein by reference thereto.

### FIELD OF THE INVENTION

The present invention relates to functional single-domains of antibody heavy chain variable regions, processes for the preparation and use of phage display libraries for identification and isolation of functional antibody single-domain molecules which bind to a desired constituent, and to pharmaceutical compositions containing the selected binding molecules.

### BACKGROUND OF THE INVENTION

The specificity of the immune system is dictated by a very large repertoire of molecular surfaces that are clustered within two homologous families of proteins: antibodies and the T cell receptors. The two families share structural homology and have similar function, i.e. to confer specificity in antigen recognition (reviewed by Padlan, Mol. Immunol., 31, 169-217, 1994).

The intact native antibody molecule generally contains heterodimeric structures of heavy and light chains, interconnected by disulfide bridges. Antigen recognition is conferred on the antibody by a limited number of hypervariable surface loops, differing in sequence and in length between different antibodies, and which are connected to a conserved framework structure. Both heavy and light chain variable regions each contain three hypervariable loop domains also referred to as Complementarity Determining Regions (CDRs). The three CDRs are designated as CDR1-CDR3 and are encoded by the recombined variable region gene segments.

Understanding recognition of antigen targets at the molecular level is both of fundamental and applied importance, since the ability to mimic these loops using small molecules is of important therapeutic value. In addition to the design of novel reagents that are based on antibody hypervariable loops, small and recombinant versions of antibodies are of fundamental importance in the field of targeted therapy and imaging (reviewed by Reiter and Pastan, Trends in Biotech 16, 513-520, 1998).

Recent developments in antibody engineering and recombinant DNA technology have made it possible to generate recombinant antibodies with high specificity and affinity for theoretically any antigen by employing phage display technology and constructing very large repertoires of antibodies that are displayed on the surface of filamentous phage (Winter et. al., Ann. Rev. Immunol. 12, 433-455, 1994). International patent application WO 92/18619 describes methods for producing a library of DNA molecules capable of expressing a fusion polypeptide on the surface of a filamentous phage particle (phagemids) and producing heterodimeric receptors such as antibodies, and T-cell receptors.

These large repertoires of naive, immune, or synthetic antibody fragments are fused to a minor phage coat protein; they are integrated into the DNA of the filamentous phage and displayed on the phage surface. Panning and selection of individual phage clones can screen the phage population containing tens of millions of individual clones through binding to an immobilized antigen (Barbas, Nature Medicine 1, 837-839,1995).

After selection, antibody genes rescued from the phage genome can be expressed very efficiently in bacteria for the production of soluble functional recombinant antibody fragments (Ward et. al., Nature 341, 544-546, 1989).

Several forms of recombinant antibody fragments can be designed to substitute for large intact immunoglobulin molecules. These options include Fab fragments or Fv fragments that are stabilized and/or covalently linked utilizing various strategies (Bird et. al., Science 242, 423-426, 1988).

Fv fragments of antibodies are the smallest modules of antibodies that contain the functional antigen-binding moiety without significant loss in antigen affinity and specificity. U.S. Pat. No. 4,946,778 describes single chain molecules with the characteristics of antibody. These molecules are produced by converting two naturally aggregated but chemically separate light and heavy polypeptide chains from an antibody variable region into a single polypeptide chain which will fold into a three dimensional structure very similar to the original native structure. Furthermore, the single chain molecules in that disclosure may have binding specificity and affinity substantially similar to the binding specificity and affinity of the light and heavy chain aggregate variable region of an antibody.

Smaller fragments of antibodies are advantageous for pharmaceutical applications for cancer targeting and imaging for example when small antigen binding molecules are needed to penetrate into large solid tumors.

The Fv fragments of antibodies consist of the heavy chain and light chain variable domains and typically the hypervariable loops (CDRs) of both chains contribute to antigen binding. However, there are examples in which heavy chains alone retain a significant binding ability in the absence of light chain. It is also well established, from structural studies, that CDR3 of the heavy chain contributes the most to antigen binding because CDR3 residues are responsible for most of the surface contact area and molecular interaction with the antigen (Harber and Richards Proc. R. Soc. London Ser B., 166, 176-187, 1966). Little, if any, binding activity was observed for isolated light chains.

In view of these data, attempts were made to isolate single VH domains. For example VH domains were isolated from expression libraries derived from immunized mice (Ward et. al., ibid). PCT application WO 94/18219 discloses methods for producing phage display antibody libraries and for increasing antibody library diversity by inducing mutagenesis within the CDR regions. Furthermore, methods for producing binding sites within the CDR regions of immunoglobulin heavy or light chains that are displayed on the surface of filamentous phage particles are disclosed in PCT application WO 94/18221.

More recently, it has been shown that camels make functional immunoglobulins that naturally lack light chains (Hamers-Casterman et. al., Nature 363, 446-448, 1993). As a result of these findings, antigen-binding VH domains were rescued from a human phage-displayed VH library (Davies and Reichmann, Biotechnology 13, 475-479, 1995). In this case a human VH/VL interface of camelid immunoglobulin heavy chain was mimicked to prevent non-specific binding of the VH through its interface for the light chain variable domain. This was achieved through three mutations in the VH/VL interface that mimic camel heavy chains naturally devoid of light chain partners.

U.S. Pat. No. 5,702,892 (to Mulligan-Kehoe) discloses phage-display libraries of immunoglobulin single-domain heavy chains. The library disclosed is constructed in an M13-derived expression vector. The nucleotides encoding either CDR1 or CDR3 comprise a plurality of synthetically produced random nucleotides. A fusion protein that includes amino acid sequences encoded by the vector insert is expressed on the outer surface of the recombinant phage, which make up the library. The fusion proteins of the library are advantageously capable of binding a ligand. The second aspect of this disclosure relates to a method of inhibiting an activity of an intracellular constituent.

The VH sequence disclosed in U.S. Pat. No. 5,702,982 did not contain a reading frame and could not be translated into VH protein. No biochemical characterization of the produced proteins, nor data on the stability of the VH fragment were disclosed. The relevant protein was expressed only intracellularly, and there were no teachings regarding cloned proteins or peptides which are expressed without the phage.

### SUMMARY OF THE INVENTION

The present invention now provides small antibody-derived recognition units for experimental, medical, and drug design purposes. These units are provided in the form of “microbodies” which are herein defined as single-domain antibody-like polypeptides or proteins which are soluble and stable and capable of binding a specific antigen of interest. These microbodies are encoded by selected clones and are produced as proteins by any of the methods known in the prior art including but not limited to production in E. Coli as insoluble inclusion bodies and in-vitro refolding. Alternative suitable production hosts include but are not limited to additional unicellular organisms, whether prokaryotic or eukaryotic, or cell lines from multicellular organisms, whether plant or animal, the latter ranging from insect to mammalian cells.

One aspect of the present invention involves a phage-display library of a single-domain of the variable region of the heavy chain of an antibody molecule (VH). The phage display library according to the present invention is based on a natural framework scaffold of a monoclonal antibody, without any induced mutations or modifications in the original VH/VL interface framework residues, having a unique VH/VL interface comprising at least one charged residue and a randomized CDR3. A VH library according to the present invention is a valuable source for the isolation of recombinant antibody fragments of minimal size against antigens of interest.

Another aspect of the present invention is a method for the preparation of a single-domain VH phage-display library that is based on a natural framework scaffold of a monoclonal antibody with a unique VH/VL interface and a randomized CDR3. The monoclonal antibody scaffold can be from any suitable mammal, including human or humanized monoclonal antibodies. In a currently preferred embodiment, the antibody scaffold can be obtained conveniently from murine monoclonals, as exemplified herein. Whereas in most VH families residue 44 is a Glycine, cloned VH genes were screened initially for families in which position 44 is other than Glycine and a VH clone was selected that belongs to mouse VH group I (A). It was found that this VH clone has a basic lysine residue instead of the highly conserved glycine commonly found in position 44. Thus a crucial scaffold element representing the VH/VL interface in this exemplary library comprises the sequence Lysine-44, Leucine-45, and Tryptophan-47.

The present invention also provides clones which bind selectively to a specific antigen of interest, such clones being selected from the above libraries.

One preferred embodiment according to the present invention, includes polypeptides derived from the VH libraries comprising the randomized sequence in the CDR3. These polypeptides, denoted herein as microbodies, are stable monomeric single-domain antibody-like molecules, which are capable of binding a specific constituent.

Shorter peptides derived from the randomized sequence in the CDR3 represent another preferred embodiment of the invention. These peptides are stable antibody-like peptides, capable of binding a specific constituent of interest.

The microbodies according to the present invention are antibody-like molecules representing a functional monomeric single domain having a molecular weight in the range of 10-15 kD on average. Shorter peptides derived from the CDR3 loop, which retain the binding attributes of interest, are between 4-20 amino acids in length, preferably 7-15 amino acids in length.

One currently most preferred embodiment of the present invention comprises immunoglobulin-binding molecules which are either microbodies or shorter peptides.

Another most preferred embodiment of the present invention comprises microbodies or shorter peptides which are capable of binding tumor necrosis factor (TNF) which is absorbed or linked to a solid support. Yet more preferred embodiments comprises microbodies or peptides which are capable of binding membrane or cell-bound TNF. A most preferred embodiment according to the present invention provides microbodies or peptides which are capable of binding soluble TNF.

Another aspect of the present invention is directed to pharmaceutical compositions comprising as an active ingredient microbodies or peptides isolated according to the principles of the present invention.

Yet another aspect of the present invention is directed to the use of pharmaceutical compositions comprising these microbodies or peptides for production of medicaments useful for the treatment or diagnosis of diseases and disorders. The present invention discloses methods of treatment of disorders wherein TNF is involved including but not limited to inflammatory bowel disease, rheumatoid arthritis, septic shock, multiple sclerosis, chronic inflammation, and allograft rejection.

Therefore, the present invention provides pharmaceutical compositions comprising pharmacologically or diagnostically active microbodies prepared according to the methods disclosed herein and a pharmaceutically acceptable carrier or diluent. Also, methods are described for the treatment of diseases comprising administering a pharmaceutical composition comprising a therapeutically effective amount or a microbody or peptide prepared according to the principles of the present invention. Furthermore, methods for the diagnosis of diseases are provided which methods include the step of administering a pharmaceutical composition comprising a diagnostically effective amount or a microbody or peptide prepared according to the principles of the present invention.

Essentially all of the uses that the prior art has envisioned for monoclonal or polyclonal antibodies, or for fragments thereof, can be considered for the molecules of the present invention. These uses include research, diagnostic techniques and therapy.

By way of exemplification, a heavy chain variable region (VH) single-domain phage-display library was designed and constructed. The scaffold that was used for library construction was a native sequence of a monoclonal murine antibody with unique VH/VL interface. In contrast to any previously known libraries, there was no need to modify any residues in the VL interface residues to avoid non-specific binding of the VH domain. The library repertoire was generated by the randomization of 9 amino acids in CDR3, to yield a large repertoire of independent clones. The library was selected through binding to protein antigens and individual clones were isolated. Isolated polypeptides were recovered having binding affinities were obtained in the nanomolar range.

The VH genes encoding for specific binding clones were rescued and expressed in large amounts in E. coli. Large amounts of soluble and stable single-domain VH protein were made from insoluble inclusion bodies by in-vitro refolding and purification. Biochemical and biophysical characterization of the VH protein revealed a highly specific, correctly folded, and stable monomeric molecule. The properties of these molecules make them useful for clinical, industrial, and research applications as well as toward the improvement in the design of small molecules that are based on the hypervariable loops of antibodies.

### BRIEF DESCRIPTION OF THE DRAWINGS

FIG. 1 is an illustration of a composition of single-domain VH library: nucleotide and amino acid sequence.

FIG. 2 illustrates tbe construction of single-domain VH library: schematic presentation.

FIG. 3 illustrates the binding and specificity of phage clones to antigens, with

FIG. 3A showing the binding titration of isolated phage clones to TNF;

FIG. 3B showing the binding titration of isolated phage clones to Ig; and

FIG. 3C showing the binding specificity of Ig reactive phage clones.

FIG. 4 illustrates the binding specificity of microbody clone number 7 and control to immobilized TNF and non-relevant antigens.

FIG. 5 illustrates the production and purification of single-domain VH protein, with

FIG. 5A showing the SDS-PAGE analysis of purified single-domain VH protein; and

FIG. 5B showing the molecular homogeneity (by FPLC) of the refolded purified VH single-domain molecule.

FIG. 6 shows the biochemical characterization of single-domain VH protein. CD spectra of refolded single-domain VH protein.

FIG. 7 illustrates the characterization of the binding properties of Ig-specific VH single-domain, with

FIG. 7A showing the ELISA binding assay of purified Ig-specific single-domain VH protein to human IgG; and

FIG. 7B showing the competition binding analysis of VH single-domain protein to human IgG using iodinated Ig-specific VH protein and increasing concentrations of cold VH protein.

FIG. 8 illustrates the characterization of the binding properties of Ig-specific VH single-domain, with

FIG. 8A showing the binding specificity of purified Ig-specific VH protein to Ig subtypes and Ig fragments; and

FIG. 8B showing the binding analysis of Ig-specific VH single-domain protein to IgG by real-time surface plasmon resonance technology.

FIG. 9 illustrates the specific binding of single-domain VH clones to Streptavidin and other proteins as tested by ELISA.

### DETAILED DESCRIPTION OF THE PREFERRED EMBODIMENTS

The antigen binding site of antibodies is formed by the hypervariable loops of the variable domains of light and heavy chains. Residues present in all six loops, three in each domain, may be actively involved in molecular interaction with the antigen. It is well established now from structural studies involving crystallographic analysis of antigen-antibody interactions, that residues in the CDR3 of the heavy chain contribute the most to antigen binding by making most of the contacts with the antigen. It is also known in the art that camelid immunoglobulin heavy chains can occur naturally without light chains but still bind antigen.

It is now disclosed that according to the present invention it is possible to obtain native heavy chain variable region sequences, including but not limited to murine sequences, without “camelid” mutations or any other induced mutations. Mutations induced in the sequence for stabilization, as is common in the prior art, were not necessary according to the present invention.

The disclosed protein sequences derived are as stable as native intact immunoglobulins and furthermore, they retain the binding attributes of intact immunoglobulins. The sequence in the “camelid mutation” region, namely in the region which corresponds to the VH/VL interface in a native heavy chain, is unique and stable in the present invention. These and further advantages over the background art will become apparent from the description of the currently preferred embodiments of the present invention.

In principle, the present invention provides for the first time a small monomeric functional unit derived from an antibody, which can be obtained in substantially purified form as a polypeptide which is soluble and stable and retains the binding capacity to any antigen of interest. These miniature antibodies are herein denoted as microbodies.

In the specification and in the claims the term “microbodies” refers to single-domain functional modules of antibodies as described above.

In the specification and in the claims the term “polypeptide” refers to a single chain of amino acids and may also be referred to as a protein.

In the specification and in the claims the term “soluble” refers to a molecule which is present in a substantially non-aggregated, non-precipitated form in solution in aqueous medium.

In the specification and in the claims the term “stable” refers to a compound that is sufficiently robust to survive isolation to a useful degree of purity, and formulation into an efficacious therapeutic agent.

As used herein and in the claims the term “antigen” defines any given molecular entity of interest including but not limited to: protein, polypeptide, peptide, glycoprotein, carbohydrate, polysaccharide, oligosaccharide, disaccharide, lipid, lipoprotein, or any organic molecule for which it is desired to obtain a binding molecule according to the principles of the present invention.

Certain abbreviations are used herein to describe this invention and the manner of making and using it. For instance, BSA refers to bovine serum albumin; CDR refers to Complementary Determining Region; CFU refers to colony forming unit; CH refers to constant heavy chain region of antibodies; CL refers to constant light chain region of antibodies; CPM refers to counts per minute; ELISA refers to enzyme linked immuno-sorbent assay; Fab refers the portion of the immunoglobulin molecule which contains the antigen binding site, containing the VH, CHI, VL and CL domains of antibodies; FPLC refers to fast performance liquid chromatography; Fc refers to the constant portion of the immunoglobulin molecule which contains the CH2 and CH3 constant domains of antibodies; HRP refers to horseradish peroxidase; Ig refers to immunoglobulin; IgG refers to immunoglobulin gamma; PCR refers to polymerase chain reaction; SAV refers to Streptavidin; scFv refers to single chain variable region containing the VH and VL domains of antibodies; SDS-PAGE refers to sodium dodecyl sulfate polyacrylamide gel electrophoresis; SPR refers to surface plasmon resonance; TMB refers to 3′,3′,5′,5′,-tetramethylbenzidine; TNF refers to Tumor necrosis factor; VH refers to heavy chain variable domain and VL refers to light chain variable domain.

Natural coded amino acids are represented by three-letter codes or by single-letter code, according to IUPAC conventions. When there is no indication, the L isomer is used.

The nomenclature used for the residues in the VH sequence is according to the Kabat system (Kabat, et al, 1991 Sequences of proteins of immunological interest. US Public Health Services, NIH, Publication no. 91-3242).

A phage display library of VH single domain proteins was generated and used to isolate binding molecules against antigens to which the library was selected. An unmodified naturally occurring VH scaffold sequence was used as a framework for the construction of the VH library in which the CDR3 loop was randomized to create the VH repertoire.

This particular VH sequence was chosen by computer sequence analysis using information about the molecular properties and interactions that compose the VL interface. This is in contrast to previously made VH libraries in which the VL interface of the VH domain was mutated to mimic camelid heavy chain variable domains that are naturally devoid of light chain partners.

On the structural level, the interface interactions of the VH and VL canonical structures of a typical VL interface (44G, 45L, 46E, 47W) [PDB access code 2fbj and 2fb4] was compared with our VL interface (44K, 45L, 46E, 47W) [PDB access 1baf]. Significant differences in their contact residues where found, which may indicate the structural and functional differences of the two interfaces and their contribution to VH domain stability (Padlan, Mol. Immunol., 31, 169-217, 1994). The modifications in the VL interface are required to abolish non-specific binding of the single VH domains and to reduce the tendency for the formations of dimers because of the hydrophobic nature of the residues that compose the VH/VL interface.

VH domain proteins specific for the antigens TNF, Ig and Streptavidin were rescued from the VH library after expression as fusion proteins to the minor coat protein on the surface of filamentous phage. The selection process consisted of panning on polystyrene immobilized antigen.

The binding of phage clones and purified VH protein was specific. They recognized only the antigen against which they were selected. The VH domains do not exhibit non-specific binding to other ligands.

VH domains are expressed very efficiently in bacteria, they are made as insoluble intracellular inclusion bodies. More than 30 mg/liter of highly purified active VH can be produced by in-vitro refolding from shake flask cultures that are harvested at OD600 nm=2.5-3.0. The VH can be efficiently purified by standard chromatography techniques of ion exchange and size-exclusion.

The secondary structure of the VH domain is identical to that of VH domains found as part of Fv, scFv, or Fab (or generally Ig V domains). The VH domains are very stable molecules that can be kept at high concentrations for structural analysis. Their stability is similar to that obtain with other stable Ig-based recombinant molecules such as scFv and Fab fragments.

Biophysical analysis of the VH protein using analytical ultracentrifugation revealed that the VH could be maintained predominantly as a monomer. Although it has a very weak tendency to dimerize, with a dissociation equilibrium constant for dimer formation of 1.1 mM, no indications of higher oligomers were found. This suggests that high concentrations of VH protein can be achieved without stability problems that occur due to aggregation. This is the first demonstration of such analysis on VH protein.

Recently, similar results were obtained for another Ig-like domain; a unique Va domain of a recombinant T cell receptor that was produced as a very stable monomeric protein (Plaksin et. al., J. Exp. Med. 184, 1251-1258, 1996).

Previous studies indicated the VH proteins are very unstable and mutations in the VL interface were required to maintain the VH protein in a soluble and stable form. Dissociation constants that were detected for an Ig-binding VH protein were 20-100 nM. These values are similar to those determined for antibody fragments selected from synthetic scFv or Fab phage displayed naive repertoires of comparable size (Barbas ibid). The properties of the VH domain that are described here make them attractive for clinical applications.

It was demonstrated previously that small antibody fragments perform better in vivo than whole antibodies or Fab's (Colcher et. al., J. Natl. Cancer Inst. 82, 1191-1197, 1990). When used for pharmaceutical applications such as imaging or drug carriers for targeted therapy, the reduced size of the molecule enable them to penetrate faster and better into tissues. The bio-distribution and renal clearance is faster.

VH domains can be used for in-vitro and in vivo studies in the same way that other antibody fragments are being used. VH domains can be labeled with radioisotopes, fluorescent probes, or other detection markers in the same way that antibody fragments are being labeled.

Fusion proteins can be constructed with VH domains (with reporter proteins, fluorescent proteins, toxins, etc) as well as coupling to various agents.

The affinity of the selected VH domain is high enough to perform these tasks without further improvement. However, it is tempting to try and improve the affinity of the VH domain and generate second generation of improved molecules. This can be achieved by further randomization of selected residues followed by further selection. More efficient is the direct isolation of high affinity binders from the original repertoire by improvement of the library complexity.

Most desirable is the construction of large libraries, which cover more than the theoretical repertoire. These large libraries were constructed for scFv fragments of antibodies by recombination between large VH and VL repertoires (Griffiths et. al., EMBO J. 13, 3245-3260, 1994). The size of the CDR3 loop in the library can be enlarged combined with codon-based mutagenic oligonucleotides that can be used to avoid stop codons or cysteines within the CDR. Randomization of other CDRs is also desirable to construct larger repertoires. The generation of such libraries will enable also structure-function relationship studies on the relations between certain CDR3 loop sizes and the type of ligand or the relative contribution of each CDR loop for binding.

The use of VH domains for structure-function studies can be also an important tool for drug discovery. As shown hereinbelow, it is possible to select single-domain VH phage clones that bind a specific consensus sequence with specificity to polystyrene that was defined previously by a peptide phage library (Adey et. al. Gene 156, 27-31, 1995). This suggests that the main and randomized CDR3 loop in the VH single-domain phage library can replace screening of peptide libraries and be an improved alternative because the leads discovered with the single-domain library have a significantly better binding affinity compared with leads isolated from peptide libraries.

The hypervariable loops of antibodies and in particular CDR3 are sequential stretches of conformationaly constrained random amino acids. Understanding recognition at the molecular level by structural studies such as NMR and crystallography combined with molecular modeling is both of fundamental and applied importance since the ability to mimic these loops using small molecules is of broad therapeutic use. Synthetic and computerized tools can be combined for the design and synthesis of peptides designed to structurally resemble, and mimic antibody hypervariable loops. Success in this goal will lend credence to the idea that these loops can, in certain cases, mimic biological molecules and moreover, provide a powerful generic tool for the design of novel drugs (Sheriff and Constantine Nature Struct. Biol. 3, 733-736, 1996).

The functional VH domains that can be isolated from the VH library are the molecular leads that will enable the design and synthesis of novel peptides that are based on the hypervariable loop of the isolated VH sequence. Once the design principles and synthesis analysis are laid out, additional activities such as catalytic abilities, can be incorporated in the peptide molecule while conserving recognition, thus broadening the scope of the process developed. All this can be implanted into an efficient generic process leading to novel drug discovery.

Initial examples for such a process were recently demonstrated by the generation of potent enzyme inhibitors that were derived from dromedary heavy chain domains (Ghahroudi et. al. FEBS Lett. 414, 521-526, 1997). Competitive inhibitors against bovine erythrocyte carbonic anhydrase and porcine pancreatic oc-amylase were isolated. Crystallographic analysis of recombinant dromedary heavy chain single domain with lysozyme revealed the amino acids that are primarily involved in antigen recognition. They form an internal image of the lysozyme active site cavity and can inhibit the enzymatic activity in a competitive manner.

Seven consecutive amino acids of the CDR3 loop form a structural mimic of the natural carbohydrate substrate of the enzyme. Therefore, the exposed CDR3 loop of the dromedary VH in that disclosure, or generally VH domains that will be isolated from libraries, might be good candidates to serve as a lead compound for new drugs. Another example is the selection of a camelized VH domain that acts as an inhibitor of hepatitis C virus NS3 protease (Martin et al. Protein Eng. 10, 607-614, 1997). Another example arises from our findings herein that we were able to isolate a VH protein that can bind specifically Ig of different types and species. This product can be further developed as a specific reagent for detection, purification, and analysis of antibodies. This can be performed on the intact VH protein or alternatively using the CDR3 encoded peptide that is responsible for the unique binding specificity.

According to the principles of the present invention, the lower complexity of the antigen-binding site in isolated predominantly monomeric VH domains, being composed of only one randomized loop and two conserved loops versus six random loops in the Fv, reduces the complexity of choosing the optimal amino acid sequence from which to develop small molecules.

The current strategy for choosing a particular VH domain as a starting point for the construction of the VH library was to use a naturally occurring VH domain that can be used as a template without the need for modifications by mutagenesis. Several observations related to VH-VL interactions that were reported in the literature were used. First, that the relative stability of Fvs depend on the strength of the VH and VL interactions in a region known to be the VH-VL interface. This region is very highly conserved among VH and VL family groups and lies around position 44 in the heavy chain (Kabat numbering system) and position 105 of the light chain. These positions are located in a stretch of the β4-strand of VH which is next to, and runs antiparallel to, a stretch of the β9-strand of VL (Hβ4-Lβ9). In the model structure of Fvs as well as in a crystal structure of a disulfide-stabilized Fv, the closest interchain (VH-VL) contacts in the framework region occur within these stretches.

There are variations regarding these types of interactions with some Fvs being very stable to such a degree that the strong VH-VL interactions can hold the two domains together without the need for covalent stabilization such as a peptide linker or disulfide bonds (Bird et. al. ibid). In other cases the interchain interactions are not sufficient to stabilize the Fv and they need a covalent bridge or linker. It is also found in many Fvs the VH-VL interactions are so weak that even a peptide linker or other forms of stabilization does not enable the formation of a stable Fv because the VH and VL domains dissociate from each other rapidly.

Second, the use of unmodified VH as a template for the generation of small stable VH domains hypothetically was a concern because the exposed hydrophobic VL interface was likely to generate instability or result in non-specific binding during antigen selection of the phage-displayed VH library (Davies and Reichmann, ibid). Non-specific interactions through the VL interface also hamper structural studies on isolated VH domains in solution.

These observations suggest that VL interface residues which form weak VH-VL interactions will be better candidates to be used as a scaffold for the generation of a VH library because their weak interacting VL interface will abolish problems of non-specific binding during selection. These can be less hydrophobic or charged residues.

The sequence diversity of the VH-VL interface was therefore analyzed, using computer databases (Kabat database of sequences of proteins of immunological interest). According to this analysis a hybridoma clone was selected from our collection that is different in the interface sequence from the frequently found residues of both in vivo rearranged and in-vitro selected antibodies.

It was previously shown that so-called “camelid” mutations of positions 44 from Gly to Glu, 45 from Leu to Arg and 47 from Trp to Gly or to Ile, in the VH-VL interface enabled the construction of a VH library. The mutations that were introduced are based on sequence comparison with camelid immunoglobulin heavy chains, which occur naturally without light chain partners. These three residues comprise the VL interface and are known to be critical for VH-VL interactions (Davies and Reichmann ibid).

The three residues at these positions are highly conserved through different human and mouse VH families. Residues 45 and 47 are more conserved however than residue 44 in which some variability among VH families is being observed. In most VH families residue 44 is a Glycine. Initially, cloned VH genes were screened for families in which position 44 is other than Glycine and selected a VH that belongs to mouse VH group I (A). It was found that this VH clone has a basic lysine residue instead of the naturally occurring highly conserved glycine commonly found in position 44. Thus the crucial scaffold element representing the VH/VL interface in the currently most preferred VH library comprises the sequence Lysine-44, Leucine-45, and Tryptophan-47.

The library repertoire was generated by randomization of the third hypervariable loop (CDR3) of the VH. This loop typically makes most antigen contacts in antibody combining sites. The VH gene was produced by PCR using an oligonucleotide, which degenerate and randomize 9 residues in CDR3 between and inclusive residues 95 and 100 C. The last 2 residues of the CDR3 (101 and 102), were not randomized because of their high level of conservancy and their known structural role at the base of the loop (Chothia and Lesk J. Mol. Biol. 196, 904-917, 1987). Cloning sites were introduced by a second PCR which facilitated the cloning of the VH library into the phagemid vector pCANTAB5E as a fusion to the phage minor coat protein encoded by gene3. A repertoire of 4×10⁸ independent clones of VH domains was obtained following 3 ligation reactions and 30 electroporations.

The skilled artisan will appreciate that the following examples are merely illustrative and serve as non limitative exemplification of the principles of the present invention and that many variations and modifications are possible within the scope of the currently claimed invention as defined by the claims which follow.

### EXAMPLES

### Example 1

### Design and Construction of Single-domain V_{H} Library

Method:

The gene encoding the VH domain scaffold [family I(A)] originated from a mouse hybridoma specific for H-2D^{d}+RGPGRAFVTI peptide. The 5′ region of the VH gene was amplified by PCR using oligonucleotides SfiI5′ [5′-AAGGAAAAAAGGCCCAGCCGGCCGATGTCCAGCTGCAGGAGTCA GGACCGGC-3′] which introduced the SfiI cloning site and the 3′ region with NotI3′ oligonucleotide[5′-TATCAAATGCGGCCGCGACGGTGACAGTGGTCCCTTGGCCCCAGTAGTCMNN MNNMNNMNNMNNMNNMNNMNNMNNTCTTGCACAGTAATATGTGGCTGT-3′] that randomized 9 amino acids in CDR3 and introduced a NotI cloning site. One μg of PCR product was re-amplified (10 cycles) with the following oligonucleotides SfiI5′ short [5′-AAGGAAAAAAGGCCCAGCCGGCCGAT GTCC-3′] and NotI3′ short [5′-TATCAAATGCGGCCGCGACGGTGACA GTGG-3′] to avoid non-symmetric pairing of strands due to primer exhaustion. The final PCR product product was digested with SfiI and NotI and ligated into the phagemid vector pCANTAB 5 E (Amersham Pharamcia Biotech). Ligated DNA was electroporated into the E. coli strain TG1 (Gibco BRL).

Results:

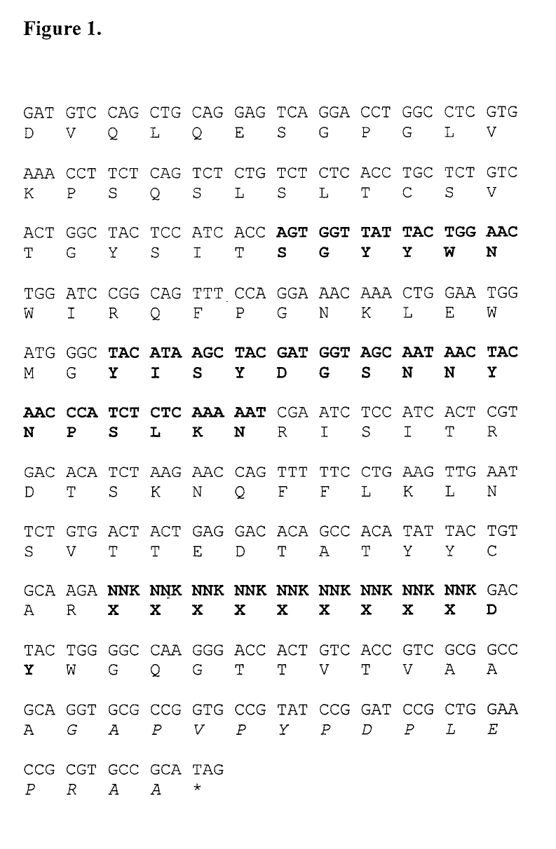

In most VH families residue 44 is a Glycine, we screened initially our cloned VH genes for families in which position 44 is other than Glycine and selected a VH that was cloned from a mouse hybridoma generated against an HIV peptide in complex with H-2D^{d}. The VH belongs to mouse VH group I (A), the nucleotide and amino acid sequence are presented in FIG. 1.

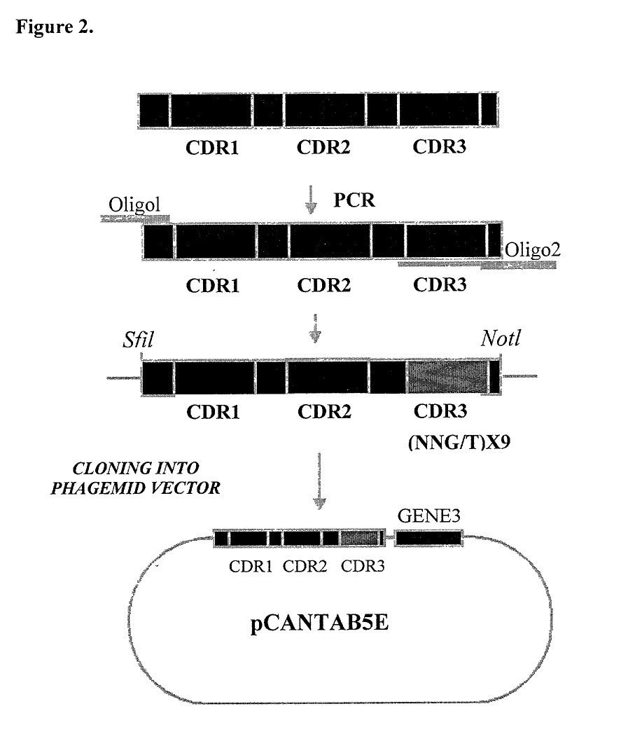

The library repertoire was generated by randomization of the third hypervariable loop (CDR3) of the VH. This loop typically makes most antigen contacts in antibody combining sites. The VH gene was produced by PCR using an oligonucleotide, which degenerate and randomize 9 residues in CDR3 between and inclusive residues 95 and 100 C. The last 2 residues of the CDR3 (101 and 102) were not randomized because of their high level of conservancy and their known structural role at the base of the loop. Cloning sites were introduced by a second PCR which facilitated the cloning of the VH library into the phagemid vector pCANTAB5E as a fusion to the phage minor coat protein encoded by gene3 (FIG. 2). A repertoire of 4×10⁸ independent clones of VH domains was obtained following 3 ligation reactions and 30 electroporations. Test screening of individual randomly picked library clones by DNA sequencing revealed an intact insert that contained the cloned VH gene and random CDR3 sequences of expected length.

### Example 2

### Panning of Library and Selection of specific Phage Clones

Method:

Phage library (5×10¹¹ cfu) was selected against antigens by panning 4 rounds on polystyrene sulfated latex beads (Interfacial Dynamics Corporation) coated with soluble TNF (R&D) or with magnetic-streptavidin-coated polystyrene beads (DYNAL) to which biotinylated Goat Immunoglobulin was immobilized. Beads were coated overnight at room temperature with 1-5 μg of protein in 50-200 μl of PBS. Following antigen immobilization the beads were blocked with PBS containing 0.05% Tween, and 5% low fat milk. Phage pool was incubated for 1 hr in blocking buffer and washed with PBS 0.05% tween. Bound phage were eluted with 500 μl of 0.2M glycine pH 2.2 and neutralized with 75 μl of 1 M tris pH 9.1.

Results:

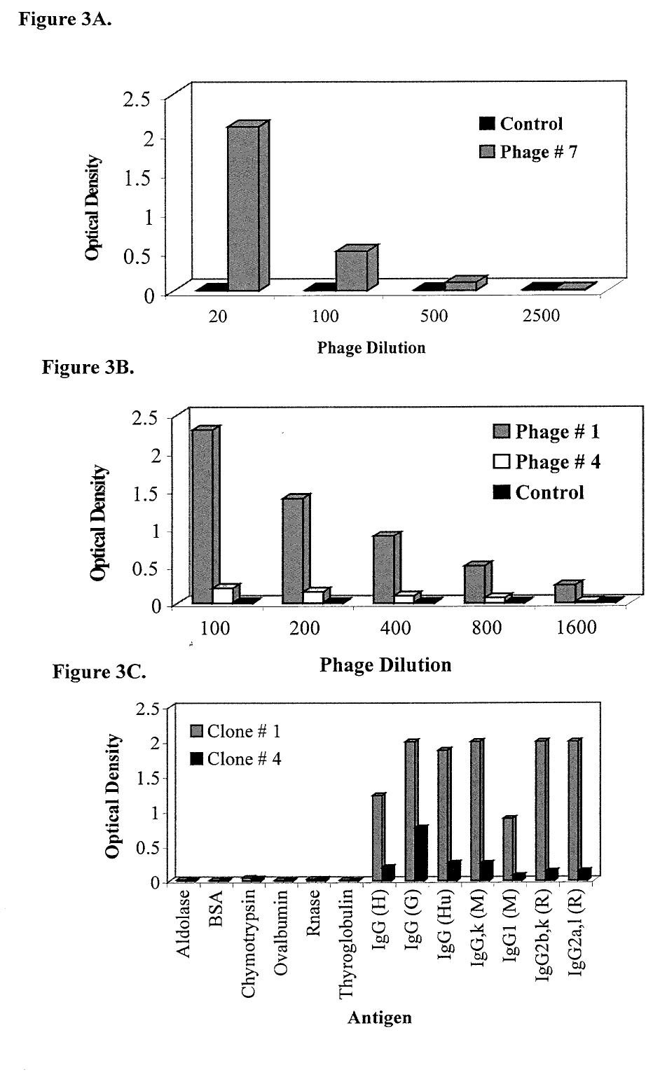

Examples for panning on two antigens, Tumor necrosis factor alpha (TNF) and Ig are shown in Table 1. Soluble recombinant TNF immobilized to sulfated polystyrene latex beads was subjected to four rounds of panning. The number of phage captured on the antigen-coated beads increased by more than 80-fold with the fourth round of panning. Forty individual phage clones from the fourth round of panning were tested in a phage ELISA assay for binding to immobilized TNF. Positive clones were sequenced. Example for phage ELISA results using individual clones are presented in FIG. 3A. We were able to isolate clones which were strongly positive and specific for TNF.

The VH library was also used in a panning experiment in which biotinylated IgG was immobilized on Streptavidine-coated magnetic beads and Ig binding phage clones were isolated. As shown in Table 1B, after four rounds of panning a 150-fold enrichment in the number of phage captured by antigen was observed. Phage ELISA of individual clones revealed strong and specific binding of the antigen compared to control phage (FIG. 3B). The genes encoding the VH protein were rescued from positive phage clones and their sequences were analyzed. As shown in Table 2, all clones exhibited an intact VH insert that contained a random 9 amino acid stretch at the expected location of CDR3. Sequence analysis revealed also consensus residues between positive clones that were isolated after the forth round of panning. For example, clones number 1 and 4 which recognize Ig show a consensus sequence of GLY-X-SER-PRO-GLN. It may be noted that in these cases X is a hydrophilic residue though this may not be a straight requirement. The difference is the location of the consensus within the CDR. For a detailed characterization of a VH single domain protein we choose to use clone 4. Consensus sequences were also obtained in several independent screenings in which antigens were immobilized on polystyrene latex beads and binding phage clones were characterized and found to be specific for plastic (polystyrene). This phenomena is characterized in the literature using peptide phage display libraries and consensus sequences rich in Trp and Tyr, which bind plastic (Adey et al. ibid). Several VH phage clones with such consensus sequences were isolated as shown in Table 2. These results demonstrate that individual antigen-binding phage clones can be isolated from the VH library. These phage clones are highly reactive in phage ELISA assays and are specific for the antigen. DNA sequence analysis of the clones isolated after the fourth round of panning revealed that the enrichment was specific for individual clones, thus, 50-60% of the sequences obtained were identical at the expected region of CDR3.

TABLE 1
Panning of single-domain V_{H} library on the antigens TNF and Ig

| | | | |
|---|---|---|---|
| Round | Phage input (cfu) | Phage output (cfu) | Enrichment (fold) |

1st. TNF

| | | | |
|---|---|---|---|
| 1 | 7 × 10¹¹ | 7 × 10⁴ | — |
| 2 | 1.4 × 10¹¹ | 2.3 × 10⁵ | 5 |
| 3 | 3 × 10¹⁰ | 3 × 10⁶ | 43 |
| 4 | 3 × 10¹⁰ | 6 × 10⁶ | 86 |

2nd. Ig

| | | | |
|---|---|---|---|
| 1 | 6.3 × 10¹¹ | 2 × 10⁴ | — |
| 2 | 8 × 10⁹ | 1 × 10⁴ | — |
| 3 | 5 × 10⁹ | 5 × 10⁵ | 25 |
| 4 | 6 × 10⁹ | 3 × 10⁶ | 150 |

TABLE 2
Amino acid composition of CDR3 region of selected phage clones.

| Amino Acid | | | | | | | | | Spe- |
|---|---|---|---|---|---|---|---|---|---|
| 1 | 2 | 3 | 4 | 5 | 6 | 7 | 8 | 9 | cificity |
| Phe | Pro | Thr | Gly | Asp | Leu | Ala | Glu | Lys | solid |
| | | | | | | | | | TNF |
| Asn | Gly | Lys | Ser | Pro | Gln | Ala | Ala | Trp | Ig |
| Gln | Ser | Gly | Gln | Ser | Pro | Gln | Ser | Ile | Ig |
| Trp | Gly | Ser | Trp | Arg | Asn | Gly | Lys | Asn | poly- |
| | | | | | | | | | styrene |
| Trp | Ala | Lys | Gly | Arg | Ser | Thr | Met | Tyr | poly- |
| | | | | | | | | | styrene |
| Trp | Gly | Met | Tyr | Arg | Ser | Gly | Thr | Gly | poly- |
| | | | | | | | | | styrene |

### Example 3

### Expression and Production of Soluble VH Single-domain Molecules

Methods:

For large scale protein production plasmid DNA from positive binding clones was re-amplified with the following oligonucleotides pET-21aVH5′NdeI [5′-GGGAATTCCATATGGATGTCCAGCTGCAGGAGTC-3′] and pET-21aVH3′XhoI [5′GGGAATTCCTCGAGCTATGCGGCACGCGG TTCCA-3′]. These inserted cloning sites that enabled subcloning into the T7 promoter-based pET-21a expression vector (Novagen). Protein was expressed at high levels in BL21 (DEλ3) cells upon IPTG induction and accumulated in intracellular inclusion bodies. Inclusion bodies were isolated and purified from the induced BL21 cells and solubilized in Guanidine HCl. Following reduction inclusion bodies were refolded in a redox-shuffling buffer system and Arginine. After refolding the protein was dialyzed and concentrated by Minisette 5K (Filtron), and purified by MonoQ (Amersham Pharmacia Biotech) ion-exchange and TSK300 gel filtration chromatography.

Results:

To produce soluble VH protein we have rescued the VH gene from the isolated phage genome by PCR and subcloned the gene into a pET system expression vector in which expression is driven by the T7 promoter. Expression of the VH genes in E Coli BL21 cells was very efficient and recombinant protein accumulated as insoluble intracellular inclusion bodies. The VH could be detected as the major band on SDS/PAGE of solubilized whole cell as well as isolated purified inclusion bodies. Purified inclusion bodies contained >90% recombinant VH protein. Although expression and production of VH was very efficient for all VH genes that were isolated (from TNF and Ig phage clones), we choose to focus on the characterization of one VH protein from phage clone #1 that recognize Ig. Inclusion bodies were purified, solubilized in 6M guanidine HCl, and refolded by in-vitro redox-shuffling buffer system.

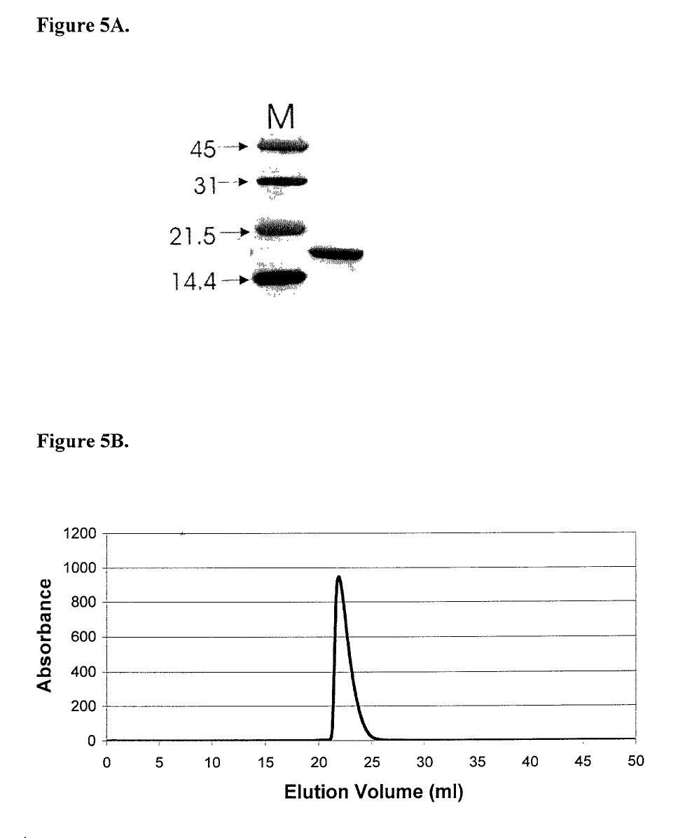

Refolded protein was purified by sequential Q-Sepharose and MonoQ ion-exchange chromatography, followed by size exclusion chromatography on TSK3000 column. The yield of refolded VH domain was 25-30%; i.e., 25-30 mg of purified soluble VH protein was obtained from 100 mg of refolded inclusion bodies. These results indicate that the VH single-domain protein can be refolded in-vitro to high yields and purity. The purified VH protein migrated as a single band with an apparent size of Mr 19300 (±5% error) on SDS/PAGE as calculated according to the relative migration against a set of molecular weight markers (FIG. 5A). Similar results were obtained when a VH protein was produced from the phage clone that binds TNF.

### Example 4

### Characterization of Binding Specificity of Phage Clones

Method:

Single phage clones were screened by ELISA assays using PEG precipitated phages. Phage or soluble VH were analyzed by ELISA in 96-well microtitre plates (Maxisorb NUNC) coated with different concentrations of antigen (0.1-1 μg/ml). Blocking of plate was performed with PBS 0.05% Tween and 5% low fat milk. Phage or protein at different concentration was added in blocking buffer. Detection second antibody was anti-M13-HRP (Amersham Pharmacia Biotech) for phage analysis and anti-E-tag-BRP (Amersham Pharmacia Biotech) for soluble protein analysis. ELISAs were developed with 3′,3′,5′,5′,-tetramethylbenzidine (TMB).

Results:

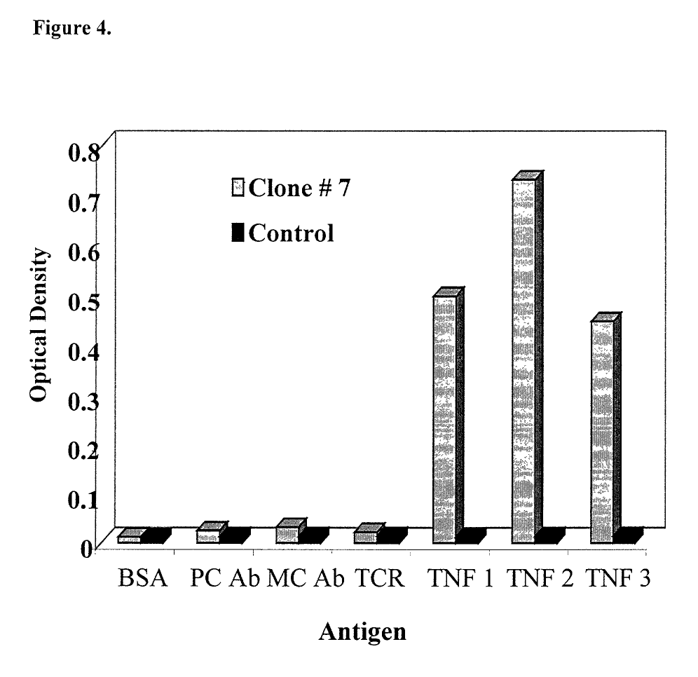

The major concern with VH single domain phage is the non specific binding due to their exposed VL interface. The reduced area of the potential antigen binding site in an isolated VH, compared with a combination of VH and VL, might also compromise specificity. We therefore analyzed by phage ELISA assays the binding of the isolated phage clones to various control antigens. As shown in FIG. 3C the isolated VH single domain phage that recognize IgG proved to be highly specific. No binding to any antigen other than that selected on, was detected. Similar specificity studies were performed on the phage clones that recognize TNF and similar results were obtained (FIG. 4). The phage clones that were isolated by panning on IgG recognized specifically a range of immunoglobulins from different species including hamster, human, mouse, and rabbit IgG. They also recognize different Ig isotypes such as IgM, IgGl, IgG2a and IgG2b. Similar results were obtained when soluble VH single-domain protein was made from the periplasm of phage clones 1 and 4. These results suggest phage clones isolated form the VH library can bind very specifically to the antigen to which they were selected for and they are not sticky. Analysis of the binding characteristics of soluble, purified VH proteins that were generated from the isolated phage clones further indicate the specificity results that were obtained with the parental phage clones.

### Example 5

### Biochemical and Biophysical Characterization of VH Single-domain Molecules.

Methods:

Mass Spectroscopy and Circular Dichroism (CD):

CD spectra of VH domain was measured in a spectropolarimeter (JASCO 500) with sensitivity of 0.5 mdeg/cm and scan speed of 10 nm/min at room temperature. Protein concentration was 1.8 mg/ml. Secondary structure calculations were measure using CONTIN, K2d and a published program.

Analytical Ultracentrifugation:

Sedimentation equilibrium experiments were conducted with a Beckman Optima XL-A analytical ultracentrifuge equipped with absorbance optical system. The protein was dissolved in PBS, and epon double-sector centerpieces equipped with quartz windows were filled with 180 μl of protein at several different loading concentrations, ranging from 1.84 μM to 91 μM. Prior to the ultracentrifugation, aggregation state of the samples was assessed using a DynaPro dynamic light scattering instrument (Protein Solutions, Charlottesville, Va.). Using an An50-Ti rotor. The samples were then centrifuged at rotor speeds of 20,000 rpm, 25,000 and 30,000 rpm at a temperature of 4° C. Absorbance distributions were recorded at wavelength of 230 nm, 250 nm, and 280 nm in radial increments of 0.001 cm, taking 50 measurements per step. Sedimentation velocity experiments were performed with the same sample after the equilibrium experiment, using a rotor speed of 50,000 rpm at 4° C., scans were taken at 250 nm or 230 nm. Sedimentation equilibrium analysis was performed by global analysis of several data sets obtained at different loading concentrations and different rotor speeds. The partial specific volume of 0.716 ml/g at 4° C., and the molar extinction coefficient of 42400 was calculated on the basis of the amino acid composition. The extinction coefficients at 230 nm and at 250 nm were determined by analysis of the signal ratios 230/280 and 250/280. Each data set was decomposed into a sum of the well-known sedimentation equilibrium exponentials for a monomer and dimer in reversible self-association equilibrium. The absence of thermodynamic non-idealities and pressure effects was assumed.

Results:

As shown in FIG. 5A the VH protein was highly pure and homogeneous as judged by SDS/PAGE. Size exclusion chromatography on a calibrated TSK3000 column showed that the purified VH preparations eluted as monomers with a molecular mass of ˜19 kDa (FIG. 5B). Mass spectrometry analysis revealed that the purified VH preparation has the expected mass. When purified protein was concentrated by ultrafiltration to levels as high as ˜2 mM (25 mg/ml) and analyzed for molecular from on size exclusion TSK3000 column in PBS, no indication of dimer formation or other stability problems, such as aggregation, were observed.

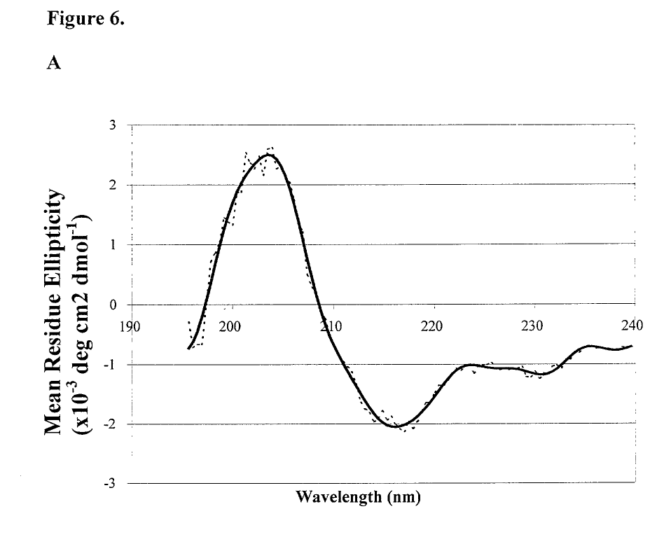

To test the stability of the VH protein, aliquots at a concentration of 0.1 mg/ml were incubated at 37° C. for up to 24 hours and subsequently analyzed for the molecular form of the VH by size-exclusion on TSK3000 column. The VH was very stable and no indication for dimerization or aggregation was observed after 24 hours of incubation at 37° C. To evaluate the nature of the secondary structure of the refolded VH domain, protein purified through both ion-exchange and size-exclusion chromatography was examined by CD spectroscopy (FIG. 6). Spectra of the VH protein showed a characteristic minima at 215 nm consistent with a largely β sheet structure. The spectra was analyzed for secondary structural calculations and found to give a specific pattern of secondary structure.

The VH showed 56% β sheet and 39% β turn, no α helix. The estimated error is 6%. This spectra is similar to those reported for a single Vα domain of the T Cell receptor (Plaksin, et. al. ibid) and to those described for single-chain Fv. These all have similarities to the spectra of Ig V domains and Fab fragments. These observations suggest that the VH protein is folded correctly after the in-vitro renaturation process of the bacterial inclusion bodies. The VH protein exhibits a CD spectrum consistent with being a single-Ig-domain. The results suggest that the VH domain is folded in the correct conformation and is similar to other Ig domains with known CD spectra. To clearly demonstrate that the VH protein is a predominantly monomeric we performed analytical ultracentrifugation. The sedimentation equilibrium profiles of the VH protein were measured over a 50-fold range of protein loading concentrations and at 3 different rotor speeds. These profiles could not be satisfactory globally fitted on the basis of a single monomeric species with the molar mass and partial specific volume predicted by the amino acid composition. Independent analysis of the sedimentation profiles at the highest and lowest loading concentrations with the floating molar mass led to a best-fit molar mass increasing with concentration. This indicated that the protein exhibited a weak self-association. Global analysis of all the equilibrium data using the predicted buoyant molar mass and considering reversible dimerization resulted in a fit of high quality, with a best-fit association constant (Ka) for dimer formation of 900±100 M⁻¹. This result indicates that the VH protein has a very low weak tendency of dimerization. Sedimentation velocity experiments were well-described by the Lamm-equation model for rapid monomer-dimer equilibria, with a best-fit monomer sedimentation coefficient of 1.7S. These results also correlate well with dynamic light scattering data, which lead to a hydrodynamic radius of 2.0 nm. No indications of higher aggregates were found in sedimentation equilibrium, sedimentation velocity, and light scattering experiments. The association constant obtained corresponds to a dissociation equilibrium constant 1.1 mM. This would suggest that at a concentration of 1 mM (˜17 mg/ml) 50% of the VH protein is in the form of a dimer and 50% in the form of a monomer. These results are the first demonstration of such biophysical analysis of a VH protein using analytical ultracentrifugation.

### Example 6

### Characterization of the Binding Properties of V_{H} Single-domain Molecules

Methods:

Binding Assays:

For competition binding assays VH protein was iodinated using the Chloramine-T method. 96-well microtiter plates were coated with 1 μg/ml human IgG (overnight, 4° C.). Plates were blocked for 1 hr at room temperature with PBS containing PBS 0.05% Tween and 5% low fat milk. Increasing concentrations of cold VH protein were added (competitor) with 2×10⁵ CPM of iodinated VH protein. Each experimental point was performed in triplicates. Binding was for 1 hr at room temperature. Plates were than washed 4 times with PBS containing 0.05% Tween. Bound Labeled VH protein was eluted from the plate by 1% SDS and 100 mM phosphoric acid. Bound and unbound (wash) were counted in a γ-counter. Non specific binding was determined by using 30-fold molar excess of cold competing VH protein. Maximal binding was determined by using iodinated VH protein without competitor.

Surface Plasmon Resonance (SPR) Analysis:

The direct binding of the expressed, purified microbody to an IgG2a murine monoclonal antibody was measured by surface plasmon resonance using the BlAcore 2000. Purified monoclonal antibody 34-2-12 (an IgG2a, K mAb with specificity for the MHC class I molecule, H-2Dd) was covalently coupled to a CM-5 carboxymethylated dextran chip using standard coupling procedures. The microbody was passed over the chip in standard HBST buffer at a flow rate of 10 ul/min, and data was collected and analyzed with the global curve fitting programs of BIEvaluation 3.0 (Biacore AB).

Results:

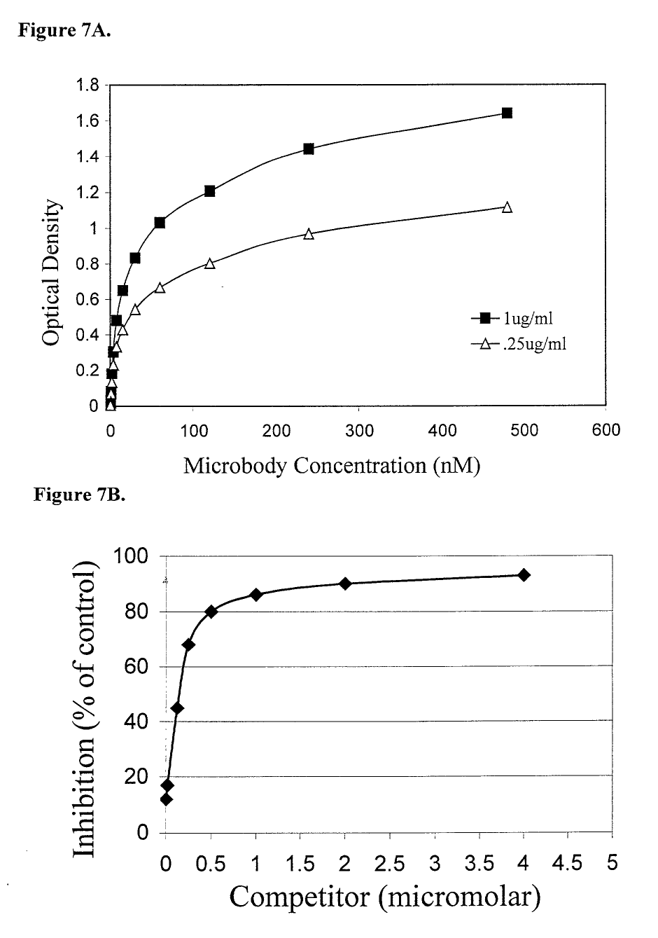

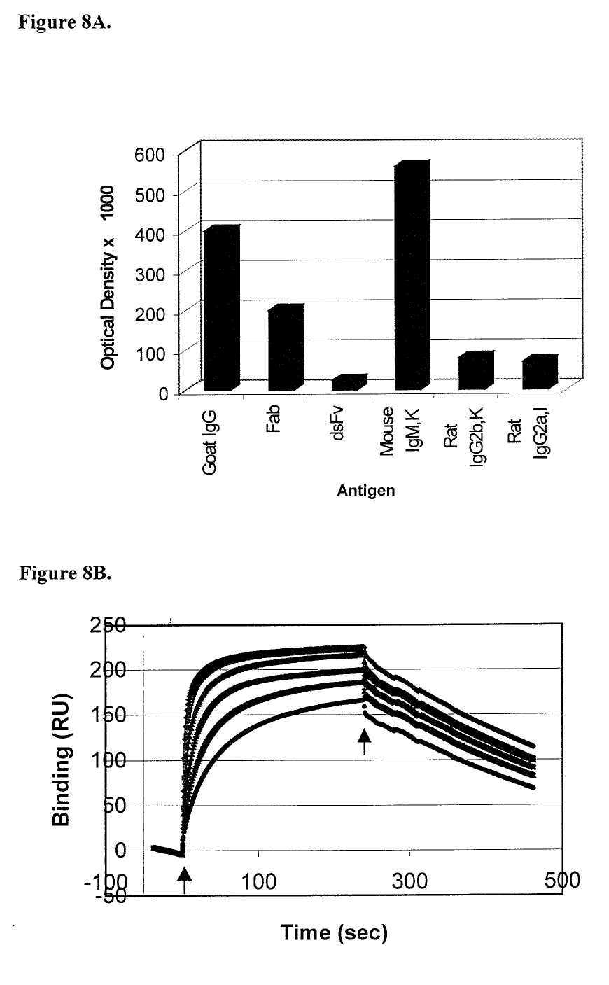

To determine the binding properties of the purified VH protein we performed several studies which assay the binding to antigen directly or by a secondary reagent in an indirect test. First, an ELISA assay was performed to titrate the binding of the VH protein to human IgG which is immobilized onto maxisorb ELISA plates. This is an indirect assay due to the fact that binding is being monitored by a secondary peroxidase-labeled antibody directed to the E-tag sequence at the carboxy terminus of the VH protein. As shown in FIG. 7A the VH binds human IgG in a dose dependent manner and VH protein concentrations as low as 1.7 ng/ml (100 pM) could be detected. When tested for specificity, the purified VH protein recognized a large variety of Ig's from different species and different isotypes. The results demonstrate that the VH recognizes specifically Ig's and that this recognition lies in the CH1 or CL domains since the VH protein recognized a Fab fragment but not an Fv fragment (FIG. 8A). No binding was detected on control antigens. To determine the binding affinity of the purified VH protein to its antigen we performed two types of binding assays. First, we performed a competition binding analysis with radiolabeled VH protein and second using real-time surface plasmon resonance (SPR) technology. In the competition binding analysis the iodinated VH protein was used as a tracer with increasing concentrations of competing unlabeled purified VH and tested for binding to immobilized human. Apparent binding affinity was ˜100 nM at which 50% inhibition of binding of iodinated VH protein had occurred (FIG. 7B). For the real-time surface plasmon resonance measurements we efficiently coupled a mouse IgG monoclonal antibody to the dextran matrix of the biosensor by random coupling through free amino groups. To estimate the kinetic association and dissociation rate constants of the VH protein for the IgG, we injected homogeneously loaded, highly purified VH single-domain protein at different concentrations over an IgG surface (FIG. 8B). By curve fitting to the dissociation (washout) phase of the binding curve, we determined the kinetic dissociation rate constant to be kd=4.13×10⁻³ s⁻¹ corresponding to a t1/2 of 168 s. The association rate constant was determined to be ka=2.14×10⁵ M⁻¹·s⁻¹. These values give a calculated equilibrium constant for dissociation, Kd=kd/ka of 1.9×10⁻⁸ M. These results correlate with the results obtained in the competition binding assay and indicate that the VH protein has a good binding affinity to its IgG antigen.

### Example 7

### Selection of Phage Clones Specific for Streptavidin and Characterization of Their Binding Specificity.

Method:

Panning was performed as described in example 2, with magnetic-streptavidin-coated polystyrene beads (DYNAL)

Results:

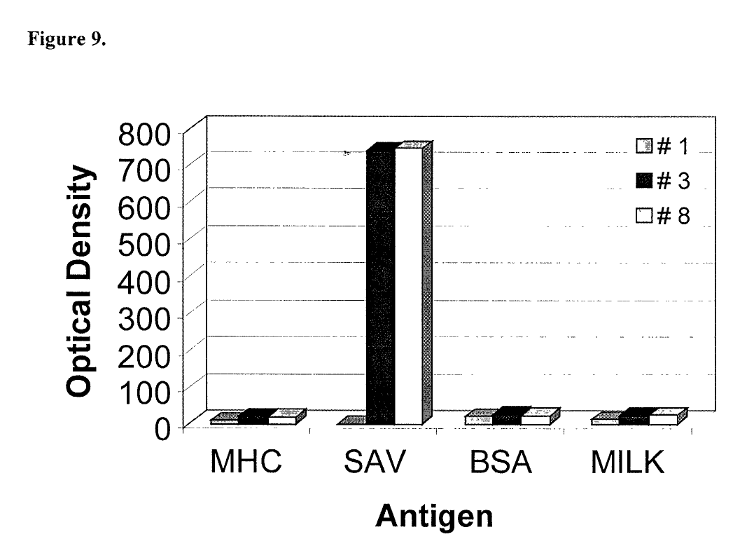

After three cycles of panning, several phage clones were selected for binding to Streptavidin. Two clones were positive by ELISA as demonstrated in FIG. 9, which shows that these phages bind Streptavidin and no other protein tested. The entire VH region of these phage clones was sequenced. The nine random amino acids of CDR3 that are encoded by the DNA of these clones are described in Table 3.

TABLE 3
Amino acid composition of CDR3 region of phage clones specific to
Streptavidin.

| Clone | 1 | 2 | 3 | 4 | 5 | 6 | 7 | 8 | 9 |
|---|---|---|---|---|---|---|---|---|---|
| 3 | His | Ala | Gln | Arg | Arg | Pro | Trp | Ile | Arg |
| 8 | Glu | Asp | Pro | His | Pro | Gln | Arg | Gly | Tyr |

Interestingly, three amino acids of the sequence of clone No. 8 (His-Pro-Gln) are identical to the consensus sequence previously published which is specific to Streptavidin (Devlin et al. Science 249, 404-406, 1990). Various improvements of the binding affinity of the His-Pro-Gln sequence were made by several investigators by elongation of the binding sequence (Schmidt and Skerra Protein Engineering 6, 109-122, 1993), and peptide cyclization (Giebel et al. Biochemistry 34, 15430-15435, 1995). Identification of the His-Pro-Gln consensus sequence as one of the Streptavidin binding motifs from our libraries of single-domains antibody heavy chain variable regions prove that these libraries are a powerful tool for identification of functional antibody single-domain molecules which bind to a desired constituent.

Based on preliminary experiments the binding affinity of the clones identified by us is higher that the previous described sequences. This may be a result of the antibody frame in which the sequences are located or the sequences themselves.

What is claimed is:

1. A polypeptide comprising a single-domain of the variable region of the heavy chain of an antibody molecule, which is soluble and stable and capable of binding a specific antigen of interest, said polypeptide comprising a natural framework scaffold of a mammalian monoclonal antibody without induced mutations or modifications in the original VH/VL interface framework residues, said VH/VL interface comprising at least one charged residue.

2. The polypeptide of claim 1 wherein the polypeptide is substantially monomeric.

3. The polypeptide of claim 1, wherein the polypeptide is encoded by a polynucleotide isolated from a phage clone selected from a phage-display library comprising a plurality of recombinant phage, each of said recombinant phages having an expression vector encoding a single-domain of the variable region of the heavy chain of an antibody molecule comprising a natural framework scaffold of a mammalian monoclonal antibody without induced mutations or modifications in the original VH/VL interface framework residues, having a unique VH/VL interface comprising at least one charged residue and a randomized CDR3.

4. The polypeptide of claim 3 wherein the selected clone is produced in E. coli as insoluble inclusion bodies and the isolated polypeptide is subsequently refolded in-vitro and purified.

5. The polypeptide claim 3 wherein the scaffold element representing the VH/VL interface comprises the sequence Lysine-44, Leucine-45, and Tryptophan-47.

6. The polypeptide of claim 1 wherein the specific antigen of interest is an immunoglobulin molecule.

7. The polypeptide of claim 3 wherein the CDR3 sequence between residues 95 and 100 C comprises the consensus sequence: Gly-X-Ser-Pro-Gln, wherein X represents any amino acid.

8. The polypeptide of claim 3 wherein the CDR3 sequence between residues 95 and 100 C is selected from the sequences: Gln-Ser-Gly-Gln-Ser-Pro-Gln-Ser-Ile, and Asn-Gly-Lys-Ser-Pro-Gln-Ala-Ala-Trp.

9. The polypeptide of claim 1 wherein the specific antigen of interest is tumor necrosis factor.

10. The polypeptide of claim 9 wherein the CDR3 sequence between residues 95 and 100 C comprises the sequence: Phe-Pro-Thr-Gly-Asp-Leu-Ala-Glu-Lys.

11. The polypeptide of claim 1 wherein the specific antigen of interest is Streptavidin.

12. The polypeptide of claim 11 wherein the CDR3 sequence between residues 95 and 100 C is selected from the sequences: His-Ala-Gln-Arg-Arg-Pro-Trp-Ile-Arg, and Glu-Asp-Pro-His-Pro-Gln-Arg-Gly-Tyr.

13. A peptide capable of binding a specific antigen of interest, said peptide being derived from the randomized sequence of the CDR3 region of a polypeptide comprising a single-domain of the variable region of the heavy chain of an antibody molecule, which is soluble and stable and capable of binding said specific antigen of interest, said polypeptide comprising a natural framework scaffold of a mammalian monoclonal antibody without induced mutations or modifications in the original VH/VL interface framework residues, said VH/VL interface comprising at least one charged residue.

14. The peptide of claim 13 wherein the polypeptide is encoded by a polynucleotide isolated from a phage clone selected from a phage-display library comprising a plurality of recombinant phage, each of said recombinant phages having an expression vector encoding a single-domain of the variable region of the heavy chain of an antibody molecule comprising a natural framework scaffold of a mammalian monoclonal antibody without induced mutations or modifications in the original VH/VL interface framework residues, having a unique VH/VL interface comprising at least one charged residue and a randomized CDR3.

15. The peptide of claim 13 wherein the peptide comprises 4-20 amino acids.

16. The peptide of claim 13 wherein the peptide comprises 7-15 amino acids.

17. The peptide of claim 13 wherein the specific antigen of interest is an immunoglobulin molecule.

18. The peptide of claim 13 wherein the specific antigen of interest is tumor necrosis factor.

19. A pharmaceutical composition comprising as an active ingredient the polypeptide of claim 1, and a physiologically acceptable diluent or carrier.

20. A pharmaceutical composition comprising as an active ingredient the peptide of claim 13, and a physiologically acceptable diluent or carrier.

21. A phage-display library comprising a plurality of recombinant phage, each of said recombinant phage having an expression vector encoding a single-domain of the variable region of the heavy chain of an antibody molecule comprising a natural framework scaffold of a mammalian monoclonal antibody without any mutations or modifications in the original interface framework residues, having a unique VH/VL interface comprising at least one charged residue, and a randomized CDR3.

22. The phage display library of claim 21 wherein the single-domain variable region of the heavy chain is substantially monomeric.

23. The phage display library of claim 21 wherein the scaffold element representing the VH/VL interface comprises the sequence Lysine-44, Leucine-45, and Tryptophan-47.

24. An isolated phage clone which binds selectively to a specific antigen of interest, said clone being selected from a phage-display library comprising a plurality of recombinant phage, each of said recombinant phage having an expression vector encoding a single-domain of the variable region of the heavy chain of an antibody molecule comprising a natural framework scaffold of a mammalian monoclonal antibody without any mutations or modifications in the original interface framework residues, having a unique VH/VL interface and a randomized CDR3.

25. The isolated phage clone of claim 24 wherein the phage display library comprises a scaffold element representing the VH/VL interface comprising the sequence Lysine-44, Leucine-45, and Tryptophan-47.

26. The isolated phage clone of claim 24 encoding a polypeptide wherein the specific antigen of interest is an immunoglobulin molecule.

27. The isolated phage clone of claim 24 encoding a polypeptide wherein the specific antigen of interest is tumor necrosis factor.

28. A method of treatment of a disease comprising administering to a patient in need thereof a pharmaceutical composition comprising as an active ingredient a therapeutically effective amount of the polypeptide of claim 1.

29. A method of diagnosis of a disease comprising administering to a patient in need thereof a pharmaceutical composition comprising as an active ingredient the polypeptide of claim 1 in an amount which is effective to diagnose the disease.

30. A method of treatment of a disease comprising administering to a patient in need thereof a pharmaceutical composition comprising as an active ingredient a therapeutically effective amount of the polypeptide of claim 13.

31. A method of diagnosis of a disease comprising administering to a patient in need thereof a pharmaceutical composition comprising as an active ingredient the polypeptide of claim 13 in an amount which is effective to diagnose the disease.

32. A method of treatment of a disease comprising administering to a patient in need thereof a pharmaceutical composition comprising as an active ingredient a therapeutically effective amount of the polypeptide of claim 9.

33. A method of diagnosis of a disease comprising administering to a patient in need thereof a pharmaceutical composition comprising as an active ingredient the polypeptide of claim 9 in an amount which is effective to diagnose the disease.
